(19) United States
(12) Patent Application Publication
(10) Pub. No.: US 2010/0159528 A1
(43) Pub. Date: Jun. 24, 2010

(54) POLYMERASE STABILIZATION

(75) Inventors: Yi Liu, Plainsboro, NJ (US); Ryan Westberry, Denver, CO (US); Scott Hamilton, Phillipsburg, NJ (US)

Correspondence Address: GE HEALTHCARE BIO-SCIENCES CORP.;PATENT DEPARTMENT, 101 CARNEGIE CENTER, PRINCETON, NJ, 08540, US

(73) Assignee: GE HEALTHCARE BIO-SCIENCE CORP., PISCATAWAY, NJ (US)

(21) Appl. No.: 12/601,102

(22) PCT Filed: Jun. 12, 2008
(86) PCT No.: PCT/EP08/57404

§ 371 Date: Dec. 2, 2009

Related U.S. Application Data
(60) Provisional application No. 60/943,667, filed on Jun. 13, 2007.

(51) Int. Cl. C12P 19/34; C12N 9/96

(52) U.S. Cl. 435/91.2; 435/188

(57) ABSTRACT

The present invention relates to methods and compositions for providing purified thermostable enzymes, particularly thermostable DNA polymerases, that are free of exogenous detergents. The present invention also provides methods for providing such purified thermostable DNA polymerases to assays in an active form by adding one or more detergents. The present invention further provides compositions and kits comprising purified thermostable DNA polymerases for use in a variety of applications, including amplification and sequencing of nucleic acids.

### CROSS-REFERENCE TO RELATED APPLICATIONS

This application is a filing under 35 U.S.C. § 371 and claims priority to international patent application number PCT/EP2008/057404 filed Jun. 12, 2008, published on Dec. 18, 2008, as WO 2008/152102, which claims priority to U.S. provisional patent application No. 60/943,667 filed Jun. 13, 2007; the entire disclosure of which is incorporated herein by reference in its entirety.

### BACKGROUND OF THE INVENTION

The present disclosure relates to thermostable DNA polymerases, compositions and kits comprising thermostable DNA polymerases, and methods for isolating and using thermostable DNA polymerases.

DNA polymerases are enzymes that catalyze the template-directed synthesis of DNA from deoxyribonucleotide triphosphates. Typically, DNA polymerases (e.g. DNA polymerases I, II, and III in microorganisms; DNA polymerases α, β or γ, in animal cells) direct the synthesis of a DNA strand from a DNA template; however, some DNA polymerases (referred to generally as “reverse transcriptases”) direct the synthesis of a DNA strand from an RNA template. Generally, these are recognized by the IUPAC-IUBMB Joint Commission on Biochemical Nomenclature under the Enzyme Commission numbers EC 2.7.7.7 and EC 2.7.7.49. Extensive research has been conducted on isolation and characterization of DNA polymerases from various organisms, including bacteria, yeast, and humans, particularly for use in in vitro reactions.

When selecting a DNA polymerase for use in a particular in vitro reaction, the skilled artisan must consider a number of variables. For example, a DNA polymerase may be selected to have its natural 5′-3′ or 3′-5′ exonuclease activity deleted (e.g. by mutagenesis or by post-translational modification such as enzymatic digestion), to exhibit a low error rate, to exhibit high processivity and elongation rate, and/or to exhibit advantageous thermal stability. The identification of DNA polymerases from thermophilic microorganisms, and the use of thermostable DNA polymerases in methods such as PCR, have led to a revolution in the ability to identify and manipulate DNA. A number of thermostable DNA polymerases have been isolated from thermophilic eubacteria, thermophilic archaea, and others.

Examples of thermostable DNA polymerases include but not limited to Taq DNA polymerase derived from Thermus aquaticus (see e.g. U.S. Pat. No. 4,889,818); Tth DNA polymerase derived from Thermus thermophilus (see e.g. U.S. Pat. Nos. 5,192,674; 5,242,818; 5,413,926); Tsp sps17 DNA polymerase derived from Thermus species spsl 7, now called Thermus oshimai (see e.g. U.S. Pat. No. 5,405,774); Pfu DNA polymerase derived from Pyrococcus furiosus (U.S. Pat. No. 5,948,663); Bst DNA polymerase derived from Bacillus stearothermophilus (U.S. Pat. No. 5,747,298); Tli DNA polymerase derived from Thermococcus litoralis (U.S. Pat. No. 5,322,785); KOD DNA polymerase derived from Pyrococcus sp. KOD1 (U.S. Pat. No. 6,033,859); nTba and Tba DNA polymerase derived from Thermococcus barosii (U.S. Pat. Nos. 5,602,011 and 5,882,904); and commercially available DNA polymerases such as THERMO SEQUENASE™ (GE Healthcare) and AMPLITAQ® (Applied Biosystems, Tabor, S. & Richardson, C. C. (1995) Proc. Natl. Acad. Sci. USA 92, 6339-6343).

Detergents have been used in the art to solubilize membranes, to enhance permeabilization effects of various chemical agents, and for disruption of the bacterial cell walls, facilitating the preparation of intracellular proteins, such as DNA polymerases, from microorganisms. Goldstein et al. discloses methods of making a thermostable enzyme which is substantially free of nucleic acids (U.S. Pat. No. 5,861,295). Gelfand et al. discloses a stable enzyme composition comprising a purifed, stable thermostable polymerase in a buffer containing one or more non-ionic polymeric detergents (U.S. Pat. No. 6,127,155). Simpson et al., Biochem. Cell Biol. 68: 1292-6 (1990) discloses purification of a DNA polymerase that is stabilized by additives such as Triton X-100.

Detergents can be difficult to remove completely from the resulting purified species. Additionally, in enzymatic reactions, such as DNA sequencing reactions, the presence of detergents may affect results. See e.g. Ruiz-Martinez et al., Anal. Chem. 70: 1516-1527, 1998. Additionally, some thermostable DNA polymerases may substantially decrease in activity over time in the absence of detergents. See e.g. U.S. Pat. No. 6,127,155 which discloses thermostable DNA polymerase in non-ionic polymeric detergents. TWEEN® 20 is one particular detergent disclosed.

U.S. Pat. No. 6,242,235 discloses the addition of a cationic surfactant based on polyethoxylate amines for DNA polymerase stabilisation.

### SUMMARY OF THE INVENTION

The present disclosure relates to compositions and methods that permit the control of the environment in which thermostable DNA polymerases, are purified and used. The disclosure provides thermostable DNA polymerase enzymes and the addition of anionic detergents. In particular the detergent can be polyoxyethylene alkyllphenyl ether, phosphates, for example, polyoxyethylene nonylphenol ether, phosphate.

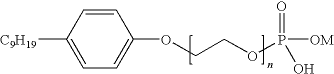

This detergent is sold under the tradename RHODAFAC™ RE-960 or polyoxyethylene alkyl ether, phosphates, for example, polyoxyethylene tridecyl ether, phosphate.

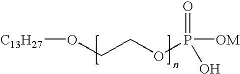

This detergent is sold under the tradename RHODAFAC™ RS-960.

Other similar anionic detergents fall into this category can be described in a more general structure as the follows:
Alkyl ethoxylated phosphate esters (I), where R is an alkyl group with C8 to C22, linear, branched, cyclic, or polycyclic hydrocarbons. The R group can also be an alkenyl group, where one or more unsaturated double bonds are in the structure. N can be from 3 to 100.
Alkyl phenol ethoxylated phosphate esters (II), where the R group is a C8 to C12 linear or branched alkyl group. N can be from 3 to 100.
Dialkyl phenol ethoxylated phosphate esters (III), where the R and R′ group is a C8 to C12 group, respectively. N is from 3 to 100.

Trialkyl phenol ethoxylated phosphate esters (IV), where R, R′, and R″ is a C8 to C12 alkyl group, respectively. N is from 3 to 100. Structure V is a special case of the IV group.

### The Chemical Structures These Phosphate Ester Surfactants Are Shown Below:

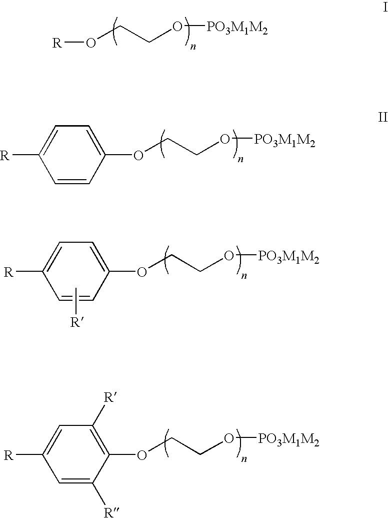

The R group can be but not limited to alkyl, arylalkyl, polycyclic, tristyril phenol. The M can be a hydrogen, ammonium, metal ion, such as sodium, lithium, and potassium.

### BRIEF DESCRIPTION OF THE FIGURES

FIG. 1 shows the PCR amplification of 1 kb, λDNA and human genomic DNA fragments in the absence and presence of detergents. Lane 1 & 7, PCR in the absence of a detergent using Tba purified without detergents; lane 2 & 8, PCR in the presence of a detergent using Tba purified without detergents; Lane 3 & 9, PCR in the absence of a detergent using Tba purified with detergents; lane 4 & 10, PCR in the presence of a detergent using Tba purified with detergents

FIG. 2 shows the screening of anionic phosphate detergents: amplification of 1 kb DNA using 0.1 or 0.01% of tested detergent. Top panel, gel-like pictures of PCR products. Lane 1, positive control (0.05% TWEEN® 20), lane 2, 0.05% RHODAFAC™ RE610, lane 3˜8, 0.01% of RHODAFAC™ RE410, RHODAFAC™ RE960, RHODAFAC™ RS960, RHODAFAC™ RS410, RHODAFAC™ RS710, and RHODAFAC™ RS610, lane 9˜12, 0.1% of RHODAFAC™ RE 710, RHODAFAC™ RE960, RHODAFAC™ RS960, and RHODAFAC™ RS710. Bottom panel, bar graph of PCR yields with each of tested detergent.

FIG. 3 shows the results of the remaining activity in lx PCR buffer after heating for 15 minutes at 95° C. Percent detergent shows final amount of detergent in the PCR reaction. For ♦, each detergent is present at the indicated amount.

♦ NP40 and TWEEN® 20

▪ RHODAFAC™-960

### DETAILED DESCRIPTION OF THE INVENTION

Preferably, thermo stable DNA polymerase I is obtained or derived from a microorganism of a genus selected from the group consisting of Thermus, Pyrococcus, Thermococcus, Aquifex, Sulfolobus, Thermoplasma, Thermoanaerobacter, Rhodothermus, Methanococcus, and Thermotoga.

The thermostable enzymes of the present invention can be obtained from any source and can be a native or recombinant protein. Thus, the phrase “derived from” as used in this paragraph is intended to indicate that the thermostable DNA polymerase is expressed recombinantly, and the expressed DNA sequence is a wild-type sequence obtained from a thermophilic organism, or a mutated form thereof. Examples of suitable organisms providing a source of thermostable DNA polymerase (sequences and/or proteins) include Thermus flavus, Thermus ruber, Thermus thermophilus, Bacillus stearothermophilus, Thermos aquaticus, Thermus lacteus, Meiothermus cuber, Thermus oshimai, Methanothermus fervidus, Sulfolobus solfataricus, Sulfolobus acidocaldarius, Thermoplasma acidophilum, Methanobacterium thermoautotrophicum and Desulfurococcus mobilis.

Preferred DNA polymerases include, but are not limited to, Taq DNA polymerase; Tth DNA polymerase; Pfu DNA polymerase; Bst DNA polymerase; Tli DNA polymerase; KOD DNA polymerase; nTba and/or Tba DNA polymerase. In certain embodiments, the thermostable DNA polymerases of the present invention have been modified by deletion, substitution, or addition of one or more amino acids in comparison to a wild-type sequence, such as Taq A271 F667Y, Tth A273 F668Y, and Taq A271 F667Y E681W.

Suitable thermostable DNA polymerase enzymes can be obtained from the organism characterised as JSER (WO 2003/004632) or Thermococcus barossii (U.S. Pat. No. 5,602,011 and U.S. Pat. No. 5,882,904).

Thermostable DNA polymerases are preferably purified from cells that either naturally express the enzyme, or that have been engineered to express the enzyme (e.g. an E. cold expressing an exogenous DNA polymerase such as Taq DNA polymerase).

In various preferred embodiments, the purification methods of the present invention comprise one or more of the following steps: (i) heating a cell lysate to denature one or more proteins; (ii) centrifuging the cell lysate to remove all or a portion of the supernatant to provide a clarified lysate; and (iii) fractionating the clarified lysate using a chromatography medium, most preferably a chromatography medium comprising a butyl functionality.

The term “thermostable” refers to an enzyme that retains activity at a temperature greater than 50° C.; thus, a thermostable DNA polymerase retains the ability to direct synthesis of a DNA strand at this elevated temperature. An enzyme may have more than one enzymatic activity. For example, a DNA polymerase may also comprise endonuclease and/or exonuclease activities. Such an enzyme may exhibit thermos/ability with regard to one activity, but not another. Preferably, a thermostable enzyme retains activity at a temperature 10 between about 50° C. and 80° C., more preferably between about 55° C. and 75° C.; and most preferably between about 60° C. and 70° C. In addition, the activity exhibited at one of these elevated temperatures is preferably greater than the activity of the same enzyme at 37° C. in the same environmental milieu (e.g., in the same buffer composition). Thus, particularly preferred thermostable enzymes exhibit maximal catalytic activity at a temperature between about 60° C. and 95° C., most preferably at a temperature between about 70° C. and 80° C. The term “about” in this context refers to +/−10% of a given temperature.

The term “active” as used herein refers to the ability of an enzyme to catalyze a chemical reaction. An enzyme will have a maximal activity rate, which is preferably measured under conditions of saturating substrate concentration and at a selected set of environmental conditions including temperature, pH and salt concentration. For the DNA polymerases described herein, preferred conditions for measuring activity are 25 mM TAPS (tris-hydroxymethyl 25 methylaminopropane sulfonic acid) buffer, pH 9.3 (measured at 25° C.), 50 mM KCl, 2 mM MgCl₂, 1 mM 2- mercaptoethanol, 0.2 mM each of dGTP, dCTP, dTTP, 0.2 mM [α-33P]-dATP (0.05-0.1 Ci/mmol) and 0.4 mg/mL activated salmon sperm DNA. The reaction is allowed to proceed at 74° C. Exemplary methods for measuring the DNA polymerase activity of an enzyme under such 30 conditions are provided hereinafter.

The term “inactive” as used herein refers to an activity that is less than 10%, more preferably less than 5%, and most preferably less than 1% of the maximal activity rate for the enzyme. For the DNA polymerases described herein, this preferably refers to comparing an activity to the rate obtained under 5 the preferred conditions for measuring activity described in the preceding paragraph. Most preferably, the thermostable enzymes of the present invention are not irreversibly inactivated when subjected to the purification steps required to obtain compositions comprising a purified thermostable enzyme free from exogenously added detergents. “Irreversible inactivation” for purposes herein refers to a loss of enzymatic activity that cannot be recovered by altering the conditions to which the enzyme is exposed.

Themostable DNA polymerases preferably are not irreversibly inactivated under conditions required for use in DNA amplification methods, such as PCR.

During PCR, for example, a polymerase may be subjected to repeated cycles of heating and cooling required for melting and annealing complementary DNA strands. Such conditions may depend, e.g., on the buffer salt concentration and composition and the length and nucleotide composition of the nucleic acids being amplified or used as primers, but typically the highest temperature used ranges 25 from about 90° C. to about 105° C. for typically about 0.5 to four minutes.

Increased temperatures may be required as the buffer salt concentration and/or GC composition of the nucleic acid is increased. Preferably, the enzyme does not become irreversible denatured at temperatures up to 90° C., more preferably up to 95° C., even more preferably up to 98° C., and most preferably up to 100° C. The 30 ability to withstand increased temperature is also often expressed in terms of a “half-life,” referring to the time at a given temperature when the enzymatic activity of a given amount of enzyme has been reduced to half of the original activity. Preferably, the enzyme has a half-life of greater than 30 minutes at 90° C.

The term “detergent” as used herein refers to surface-active agents (“surfactants”) that, when added to a liquid, reduce surface tension of the liquid in comparison to the same liquid in the absence of the detergent. See e.g. Detergents: A guide to the properties and uses of detergents in biological systems, Calbiochem-Novabiochem Corporation, 2001.

The term “purified” as used herein with reference to enzymes does not refer to absolute purity. Rather, “purified” is intended to refer to a substance in a composition that contains fewer protein species other than the enzyme of interest in comparison to the organism from which it originated. Preferably, an enzyme is “substantially pure”, indicating that the enzyme represents at least 50% of protein on a mass basis of the composition comprising the enzyme. More preferably, a substantially pure enzyme is at least 75% on a mass basis of the composition, and most preferably at least 95% on a mass basis of the composition.

In another aspect, the present disclosure provides methods for providing a purified thermostable DNA polymerase to an assay. These methods comprise adding one or more detergents to a composition comprising a purified thermostable DNA polymerase, where the composition comprising the purified thermostable DNA polymerase was previously free of exogenously added detergent. Most preferably, adding detergent to a purified thermostable DNA polymerase that was previously free of exogenously added detergent converts an inactive DNA polymerase to an active form, or increases the activity of a DNA polymerase. In various aspects, one or more detergents may be added to the compositions described above, and the resulting composition may be added to a reaction mixture for use in an assay; alternatively, a purified thermostable DNA polymerase may be added to a reaction mixture and the detergent may be added subsequently; and/or detergent may be added to a reaction mixture and the thennostable DNA polymerase may be added subsequently. In any case, the result is that a purified thermostable DNA polymerase that was previously free of exogenously added detergent is now in a composition comprising detergent.

The term “assay” as used herein refers to any reaction mixture in which a purified thermostable DNA polymerase catalyzes the template-directed synthesis of DNA from deoxyribonucleotide triphosphates or analogues such as dideoxyribonucleotide triphosphates. Preferred assays include DNA polymerase activity assays, single- or double-stranded exonuclease activity assays, single- or double-stranded endonuclease activity assays, nucleic acid amplification reactions, and nucleic acid sequencing reactions.

The anionic detergent, and in particular the anionic detergents RE-960 and RS-960 can be used in a final concentration of up to 3% (range 0.002% to 3%). Concentrations above 0.5% do not improve stability but do not decrease activity. An effective amount of anionic detergent is defined as a concentration of suitable anionic detergent which is consistent with stability and functionality of the thermostable DNA polymerase. Routine experimentation will determine what is an effective amount of any particular anionic detergent. Unless indicated otherwise concentrations are given as % W/V.

It has been observed that although DNA polymerases can be purified and stored without the presence of detergents, the polymerases do not function in the absence of detergents such as the anionic detergents mentioned previously.

### EXAMPLES

The present examples are provided for illustrative purposes only, and should not be construed as limiting the scope of the present invention as defined by the appended claims. All references given below and elsewhere in the present specification are hereby included herein by reference.

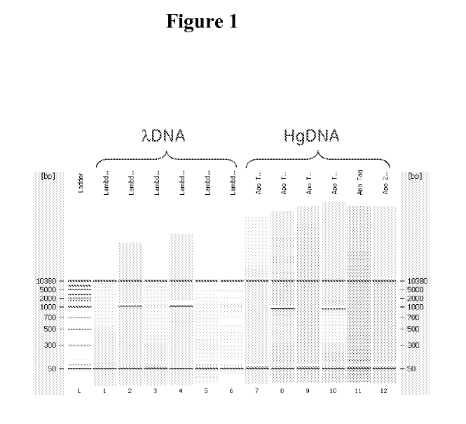

FIG. 1 shows the results of amplification of 1 kb fragments from λDNA and human genomic DNA in the presence and absence of detergent. In this experiment, two lots of DNA polymerase that were purified and stored in buffers with/without detergents were tested. Each of the tested DNA polymerase was first diluted into 20 folds in buffers with and without of TWEEN® 20, respectively, prior to PCR and then equal units of enzyme was added into PCR mix. PCRs were performed in 25 μl volume in with 200 nM of forward and reverse primers, 200 μM dNTP, 1.0 unit of Tba and 1.0 ng template DNA. 1 kb human genomic DNA and 1 kb lambda DNA fragments were amplified. Reaction buffer contains 10 mM Tris-HCl, pH 9.0, 50 mM KCl, and 1.5 mM MgCl₂. The PCR reaction was started by 2 minutes initial heating at 95° C., followed by 35 cycles with 95° C. for 30 s, 55° C. 30 s, and 72° C. for 60 s, and then final extension at 72° C. for 5 min. The PCR products were analyzed with Agilent Bioanalyzer. The results show that PCR products were only obtained from the reactions that the enzymes were diluted in a buffer containing the detergent. For those diluted in buffers without the detergent, enzymes were completely inactive and no PCR products were formed. Even for the DNA polymerase that was originally stored in the buffer having detergents, after 20-fold dilutions with the buffer that does not have the detergent, the enzyme lost activity. This data demonstrates that the detergent is required for DNA polymerase to maintain its activity.

It is known that many ionic detergents bind to protein cooperatively. The cooperative binding normally causes the denaturation of a protein. The occurrence of cooperative binding depends on the binding affinity and CMC of the detergents. The binding affinity is related the polarity of the hydrophilic head and the length of hydrophobic tail of the detergent.

Phosphate esters have relatively mild acid groups compared to strong sulphate and sulfonate based surfactants that could be too strong in affinity or polarity to cause the denaturalization of proteins. Phosphate esters are also a family of very effective surfactants for a variety of different applications. Due to the characteristic nature of phosphate esters, they are suitable surfactants for the application of polymerase stabilization.

The following 7 anionic organic phosphate detergents were tested for their ability to stabilize and activate DNA polymerase (RHODAFAC™ REs are nonyl phenol ethoxylate based phosphates and RHODAFAC™ RSs are tridecyl ethoxylated phosphates):
RHODAFAC™ RE-960
RHODAFAC™ RE-610
RHODAFAC™ RE-410
RHODAFAC™ RS-960
RHODAFAC™ RS-710
RHODAFAC™ RS-610
RHODAFAC™ RS-410

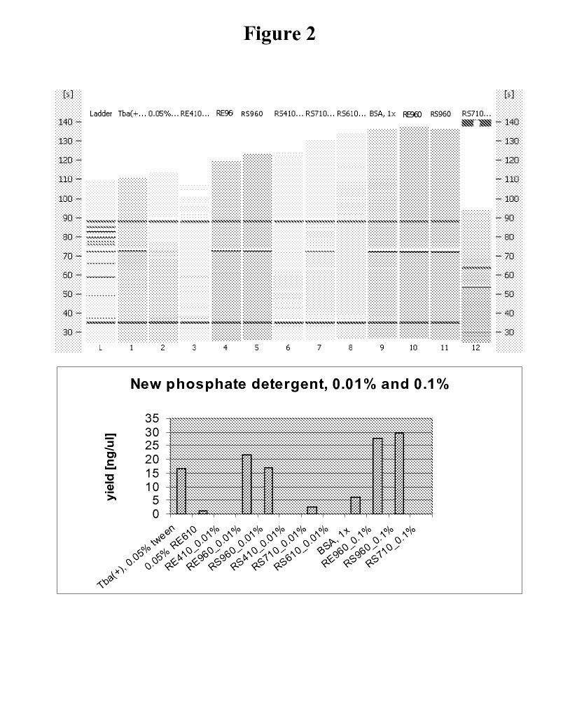

The PCR assay was used for the detergents screening. The tested detergents were first dissolved in molecular biology grade water and neutralized to pH7 with a sodium hydroxide solution to make final concentration of 5% (W/V) stock solutions. The 5% detergent solutions were then used for the screening by either adding them into enzyme dilution buffer or spiked into PCR mix. The amplification of a 1 kb λDNA fragment was used test the effect of each detergent on the Tba stability and activity. The PCR conditions are the same as described earlier. Tba that was purified and stored in the absence of detergents was used for the tests. The tested detergents were spiked into the PCR reactions at a final concentration of 0.01%, 0.05%, and 0.1%, respectively. The PCR yields were quantitated by Agilent Bioanalyzer. FIG. 2 shows part of the screening results, where detergents were added into PCR reaction mix directly at final concentration of 0.01% and 0.1%, respectively. This data shows that RHODAFAC™ RE-960 and RHODAFAC™ RS-960 work very well in stabilization and activation of DNA polymerase.

The screening results of all 7 phosphate detergents in 3 different concentrations are listed in Table 1, where the PCR yield of each PCR reaction was converted to the % activity relative to TWEEN® 20. Of the 7 anionic detergents screened, RHODAFAC™ RE-960 and RHODAFAC™ RS-960 are the best candidates in stabilization and activation of DNA polymerase. Comparing to TWEEN® 20 (the positive control), PCR yields from the reactions containing these two detergents are about 30% higher than the positive control, suggesting that these two detergents are more effective than TWEEN® 20 in the stabilization of DNA polymerase. Moreover, these two detergents work in a wide concentration range. At all three tested concentrations, they exhibit better stabilization or activation functions than TWEEN® 20 does.

The stabilization functions of these two detergents are tested for δJSER DNA polymerase as well. They show the same activation for δJSER DNA polymerase. In addition, the stabilization effects of these two detergents on DNA polymerase were also tested in the activity assay and thermostability assay. In both activity assay and thermostability test, these two detergents show the same or better stabilization effects than that of TWEEN® 20. All of the test results demonstrate that RHODAFAC™ RE-960 and RHODAFAC™ RS-960 are good replacements for the common non-ionic detergents. Using these two detergents, we can formulate storage buffer and PCR reaction buffer for DNA polymerase.

TABLE 1
Summary of anionic phosphate detergents screening results.
% Activity relative to TWEEN ® 20

| | Detergent Conc. | | |
|---|---|---|---|
| Detergent name | 0.01% | 0.05% | 0.10% |
| RHODAFAC ™ RE-960 | 130.7 | 165.8 | 167.1 |
| RHODAFAC ™ RS-960 | 101.8 | 167.9 | 178.6 |
| RHODAFAC ™ RS-410 | 0.0 | 0.0 | 0.0 |
| RHODAFAC ™ RS-710 | 15.6 | 25.2 | 0.0 |
| RHODAFAC ™ RS-610 | 0.0 | 0.0 | 0.0 |
| RHODAFAC ™ RE-610 | 70.6 | 80.3 | 36.0 |
| RHODAFAC ™ RE-410 | 0.0 | 0.0 | 0.0 |

The % stabilization activity of each detergent at each of the tested concentration was calculated using TWEEN ® 20 as a reference.

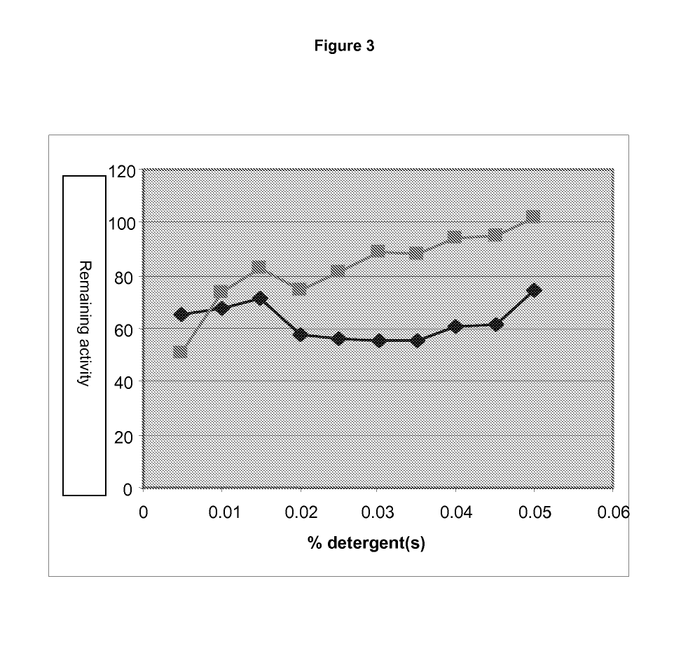

The results shown in FIG. 3 demonstrate that when a PCR reaction is performed at 95° C. in the presence of the appropriate detergent, then more DNA polymerase remains active when the PCR reaction is performed in the presence of RHODAFAC™ RE-960

1. A thermostable DNA polymerase composition comprising an anionic detergent.

2. The composition of claim 1, wherein the anionic detergent is a polyoxyethlene ether phosphate.

3. The composition of claim 2, wherein the anionic detergent is polyoxyethylene alkylphenyl ether phosphate or polyoxyethylene alkyl ether phosphate.

4. The composition of claim 1 where the anionic detergent is present at up to 0.5% concentration.

5. The composition of claim 3, where the anionic detergent is present at up to 0.5%.

6. The composition of claim 5, wherein the anionic detergent is polyethylene nonylphenol ether phosphate.

7. A method of stabilising and or enhancing DNA polymerase activity using an effective amount of an anionic detergent.

8. A method for performing PCR which comprises an anionic detergent.

9. The method of claim 8, wherein the anionic detergent is a polyoxyethlene phosphate.

10. The method of claim 7, wherein the anionic detergent is polyethylene nonylphenol ether phosphate.

11. The method of claim 8, wherein the anionic detergent is polyethylene nonylphenol ether phosphate.
